(19) United States
(12) Patent Application Publication
(10) Pub. No.: US 2010/0308498 A1
(43) Pub. Date: Dec. 9, 2010

(54) METHOD AND DEVICE FOR THE HANDLING AND THERMALLY TREATING BAG OR FILM PACKAGES

(76) Inventors: Meinhard PROTZ, Bomlitz (DE); Peter Kalisch, Bremen (DE)

Correspondence Address: FROMMER LAWRENCE & HAUG, 745 FIFTH AVENUE- 10TH FL., NEW YORK, NY, 10151, US

(21) Appl. No.: 12/651,690

(22) Filed: Jan. 4, 2010

Related U.S. Application Data
(62) Division of application No. 09/806,816, filed on Jul. 6, 2001, now abandoned.

(51) Int. Cl. B29C 71/02

(52) U.S. Cl. 264/234

(57) ABSTRACT

Method for handling and heat treatment rectangular bag or film packs with a smaller thickness than length and width, having two longer and two shorter lateral edges, characterized by an orientation of the film packs, in which one main extension plane of the film pack containing the lateral edges forms during handling and/or heat treatment an angle of less than 60° with the vertical and the longer edges are oriented substantially horizontally.

### BACKGROUND OF THE INVENTION

The invention relates to a method for handling and heat treating rectangular bag or film packs with a smaller thickness than length and width and having two longer and two shorter lateral edges, as well as a magazine-like carrier device for handling and heat treating a plurality of bag or film packs for performing said method.

In the sense of the present invention the term handling covers conveying, feeding, transferring, sorting, intermediate storing, gripping, etc.

The invention is in the field of the manufacture of bag or film packs filled with (animal) foods, in which previously manufactured (tubular) bags or film packs, which are also known as pouches, are filled with a content material in special filling and sealing devices and then closed (sealed) and are subsequently inter alia subject to sterilization before the finished products are packed.

To illustrate the aims of the invention reference is made in an exemplified manner to the production of animal foods packed in film packs where, due to veterinary regulations, a specific so-called F₀ value must be respected and which for specific products is in the range 20 to 40. For this purpose it is necessary to maintain a temperature of at least 121° C. at all points of the finished product for a specific period of time. This can be achieved, e.g. by introducing the bag packs into a steam sterilizer with a pressure between 1.5 and 2 bar.

Although the relatively flat bag packs are very favorable compared with compact shapes such as can packs with respect to the heat penetration and the time required for it. As is known the heat penetration rate increases in square law form with the greatest depth dimension of a body to be heated. The known procedure in which the bag packs or pouches are juxtaposed on tray-like plates, several plates being stacked in superimposed manner, is disadvantageous both with respect to the heat transfer in the sterilizer and also with respect to handling.

This is on the one hand due to the fact that the contact of the sterilization steam with the film packs lying on the tray-like plates is not of an optimum nature as perforated plates can only be used to a limited extent, because the perforation pattern would be transferred to the film pack materials which are relatively soft at the sterilization temperature. Also, on the other hand, because the largely manual placing of the film packs on trays represents an interruption of the otherwise substantially automated manufacturing process.

Thus, the problem of the present invention is to provide an improved method and apparatus for the handling and heat treatment of film packs or pouches. The invention solves both the problem of the partial manual handling of film packs and also the heat treatment, so that it is possible to reduce costs for the sterilization and handling of flexible film or bag packs.

### SUMMARY OF THE INVENTION

From the method standpoint, this problem is solved by a method for handling and heat treating rectangular bags or film packs with a smaller thickness than length and width, having two longer and two shorter lateral edges, which is characterized by an orientation of the film packs, in which one main extension plane of the film pack containing the lateral edges forms, during handling and/or heat treatment, an angle of less than 60° with the vertical and the longer edges are oriented substantially horizontally.

The angle is preferably less than 30°. It is even more preferred that the angle is 0°, so that the film packs are in a vertical orientation resting on a longer lateral edge.

In the case of an inclined orientation, the film packs can overlap one another in scale-like manner.

From the apparatus standpoint, the problem of the invention is solved by a magazine-like carrier device for handling and heat treating a plurality of film packs, which is suitable for performing the method according to the invention, having a plurality of successively arranged, substantially parallel partitions for forming receiving pockets for in each case two mutually spaced partitions between two adjacent receiving pockets.

Appropriately the width of the partitions substantially corresponds to the longest dimension of the film packs to be received.

The height of the partitions can be smaller than the length of the shorter lateral edge of the film packs to be received.

Preferably, the mutual spacing of the partitions forming a receiving pocket substantially corresponds to the thickness of the film packs to be received.

According to a preferred embodiment, the partitions forming a receiving pocket are slightly V-shaped with an opening angle of more than 0°.

Preferably, the partitions are flexibly held on an elongated body, so that it is possible to expand the receiving pocket, while increasing the opening angle.

Preferably, the receiving pockets have a V-shaped bottom.

Appropriately the carrier device has a stackable construction.

Preferably, the carrier device is stackable in such a way that receiving pockets of a carrier device are partly arranged in gaps between receiving pockets of a carrier device to be adjacently positioned.

### BRIEF DESCRIPTION OF THE DRAWING

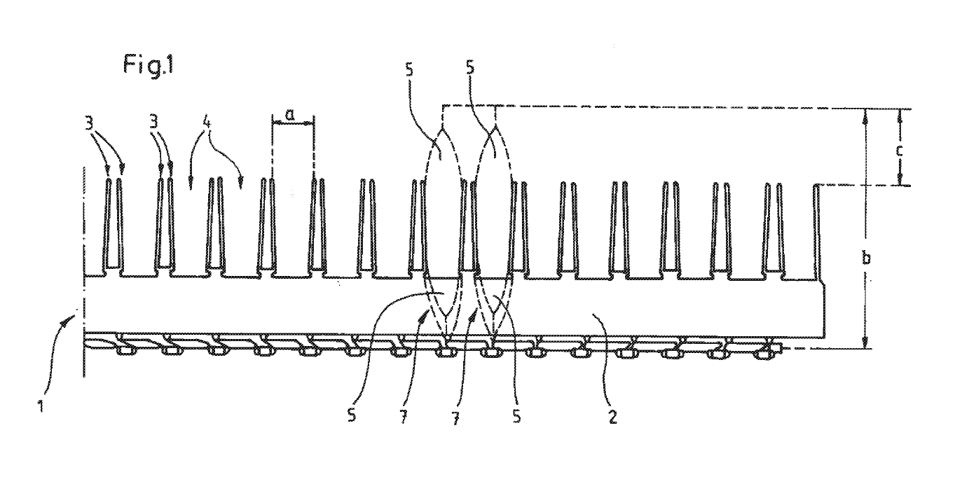

Further advantages and features of the invention can be gathered from the following description of a preferred embodiment with reference to the attached single FIG. 1, which is a detail of an inventive carrier device for handling film packs.

### DESCRIPTION OF A PREFERRED EMBODIMENT

FIG. 1 shows in side view the structure of an inventive carrier device for handling a plurality of film packs given the overall designation 1. On an elongated body 2, which can comprise a suitable profile cross-section, are fitted in parallel several rectangular partitions 3 that in each case form receiving pockets 4 between them. The mutual spacing of in each case two partitions forming a receiving pocket being chosen in such a way that it is possible to receive a single film pack 5. In each case two partitions 3 forming a receiving pocket 4 are at the top, i.e. in a direction away from the body 2, fitted in a slightly V-shaped manner, in order to facilitate the insertion or introduction of a film pack 5 from above into the receiving pocket. For example, by the fixing of the lower edge of the partitions 3 to the body 2, they are held slightly flexibly on the latter, so that it is possible to increase the opening angle of the partitions if a corresponding force is exerted thereon. This can be advantageous during a heating or sterilization process, because the content of the film pack expands by up to about 4% on heating to approximately 120° C.

As is also indicated in FIG. 1, in the vicinity of the body 2, i.e. below the partitions 3, the receiving pockets 4 are provided with a V-shaped bottom 7, on which in the case of a substantially horizontal position of the body 2 or the carrier device 1, the film packs 5 rest with their longer lateral edges.

As a result of the inventive positioning of the film packs, the width dimension of the carrier device running perpendicular to the representation plane of FIG. 1, substantially corresponds to the longest dimension of the film packs to be received, while the height b of the carrier device, which results from the height of the body 2, plus the height of the partitions 3, is smaller than the length of the shorter lateral edge of the film packs 5, so that the latter project upwards out of the receiving pockets 4, as shown in FIG. 1.

The dimensions c by which the film packs 5 are allowed to project over the upper edge of the partitions 3 is inter alia dependent on the stability of the film packs, particularly under the action of heat (softening of the film material) and should be as large as possible, so as to aid the heat transfer processes and save material with respect to the carrier device.

The method according to the invention can take place in the following way. Firstly previously produced film packs are filled and sealed and introduced into carrier devices 1 according to the invention. A large number of carrier devices are combined on pallets to block-like units and sterilized. After sterilization they are again removed from the pallets and then dried.

The film packs are then removed from the carrier devices 1 and combined into detachable and saleable pack units 20. The film packs only have to be once inserted in and once removed from the carrier devices, which can take place without manual activity and clearly facilitates handling.

The inventive handling of the film packs through their standing on their longer lateral edge in inclined or perpendicular manner leads to numerous advantages compared with a lay-flat support or an upright support on a shorter lateral edge. Firstly an automated handling is possible through the use of the inventive carrier devices, magazines or cassettes due to the fixed, predetermined spacing between two film packs. This automated handling is further accelerated by the inventive support on a longer lateral edge, because on feeding or removing shorter distances have to be covered as compared with an upright support or storage. The thickness distribution of the product contained in a film pack is also optimized with regards to heat penetration. A problem occurs here that in particular at elevated temperatures (sterilization), the film material of the packs softens and the products assume fluid-like characteristics, so that they tend to collect to a greater extent in the bottom area of a film pack, accompanied by bulging.

The bulging tendency clearly increases with the height of the film pack (static pressure of the content). This is very unfavorable for an optimum heating and/or sterilization of film packs, i.e. requiring very short heating times, where in particular in the case of animal food a specific F₀ value has to be respected, because the time required for reaching a specific temperature, under otherwise identical conditions, increases in square law form with the greatest depth dimension or thickness of the product. As a result of the handling position according to the invention these problems are largely eliminated.

The features of the invention disclosed in the description, drawing and claims are essential to the implementation of the different embodiments of the invention, both singly and in random combinations.

1. A method for handling and heat treating rectangular bags or film packs having a smaller thickness than length and width and having two longer and two shorter lateral edges comprising orienting said bags or film packs so that one main extension plane of said bags or film packs containing the lateral edges forms, during handling and/or heat treatment, an angle of less than 60° with the vertical and the longer edges are oriented horizontally.

2. A method according to claim 1 wherein the angle is smaller than 30°.

3. A method according to claim 1 wherein the angle is 0°.

4. A method according to claim 2 or 3 wherein said bags or film packs overlap one another in scale-like manner.
